(19) United States
(12) Patent Application Publication
(10) Pub. No.: US 2008/0118882 A1
(43) Pub. Date: May 22, 2008

(54) Moisture resistant pressure sensitive adhesives for orthodontic applications

(76) Inventor: Li-Hung Su, Foster City, CA (US)

Correspondence Address: ALIGN TECHNOLOGY C/O WAGNER BLECHER LLP, 123 WESTRIDGE DRIVE, WATSONVILLE, CA, 95076, US

(21) Appl. No.: 11/601,106

(22) Filed: Nov. 17, 2006

(51) Int. Cl. A61C 7/14; A61K 6/00; C08F 8/00; B32B 27/04

(52) U.S. Cl. 433/2; 523/105; 525/50; 525/329.5; 156/334

(57) ABSTRACT

A moisture resistant pressure sensitive adhesive for orthodontic application. Specifically, in accordance with one embodiment, a pressure sensitive adhesive (PSA) composition comprises a polymer base, and at least one bio-compatible hydrophobic tackifier. The PSA composition is structured such that the PSA composition is suitable for use in an oral environment.

### CROSS REFERENCE TO RELATED APPLICATIONS

The planning and fabrication of dental aligners, such as an exemplary elastic polymeric positioning appliance, is described in detail in U.S. Pat. No. 5,975,893, and in published PCT application WO 98/58596 which designates the United States, both entitled “METHOD AND SYSTEM FOR INCREMENTALLY MOVING TEETH,” both of which are assigned to the assignee of the present application.

The use of compliance indicators for monitoring orthodontic treatment compliance is described in detail in the following co-pending and commonly assigned U.S. patent application assigned to the present assignee: Ser. No. 10/949,717, entitled “SYSTEMS AND METHODS FOR DENTAL APPLIANCE COMPLIANCE INDICATION”.

### BACKGROUND OF THE INVENTION

1. Field of the Invention

Embodiments of the present invention relate in general to orthodontics. More particularly, embodiments of the present invention relate generally to pressure sensitive adhesives for orthodontic application.

2. Related Art

Orthodontic treatments involve repositioning misaligned teeth and improving bite configurations for improved cosmetic appearance and dental function. Repositioning is accomplished by applying gentle controlled forces to a patient's teeth over an extended period of time. Due to the limited space within the oral cavity and extensive movements that some teeth must undergo, the teeth will often be moved throughout a series of intermediate patterns to properly arrange the teeth. For example, molars may be temporarily moved backwards (distalized) to create adequate space for movement of the incisors. Thus, a single patient may experience an average of 25-30 stages or alignment patterns before achieving the final desired teeth arrangement.

Conventionally, repositioning of teeth has been accomplished by what are commonly referred to as “braces.” Braces comprise a variety of appliances such as brackets, bands, archwires, ligatures, and O-rings. After they are bonded to the teeth, periodic meetings with the orthodontist are required to adjust the braces. This involves installing different archwires having different force-inducing properties, or replacing or tightening existing ligatures. Between the periodic meetings with the orthodontist, the patient may be required to wear supplementary appliances, such as elastic bands or headgear, to supply additional or extraoral forces. Although conventional braces are effective, their use is often a tedious and time consuming process and requires many visits to the orthodontist's office. Moreover, from the patient's perspective, the use of braces is unsightly, uncomfortable, presents a risk of infection, and makes brushing, flossing, and other dental hygiene procedures difficult. Additionally, as conventional braces are fixedly bonded to the patient's teeth, the braces cannot be removed when the patient is eating.

The oral environment within which dental appliances are placed is a harsh environment. The mouth contains a concentration of acid formed from foods, plaque forming bacteria, and mouth secretions. Additionally, saliva generated by and present in the mouth is used to neutralize the acid through the control of pH and bicarbonate concentration. As such, the oral environment is a moist environment due to the constant generation of saliva used to control acidity.

This oral environment is not conducive to attaching objects to dental appliances. Conventional adhesives used to attach the objects are subjected to long exposure to the oral fluids present in the mouth. Conventional adhesives do not have good oral fluids resistance properties and tend to peel off from the dental appliance over time due to softening of the adhesive, for example.

Additionally, most commercially available medical-grade adhesives and adhesive tapes are not specifically designed for oral and orthodontic application. More specifically, most are specifically designed for skin care application (e.g., bandages, patches, etc.). As such, these conventional adhesives and adhesive tapes may not be biocompatible and are unsuitable for use in an oral environment.

### SUMMARY OF THE INVENTION

Accordingly, various embodiments of the present invention disclose moisture resistant pressure sensitive adhesives for orthodontic applications. Specifically, in accordance with one embodiment, a pressure sensitive adhesive (PSA) composition comprises a polymer base, and at least one bio-compatible hydrophobic tackifier. The PSA composition is structured such that the PSA composition is suitable for use in an oral environment.

### BRIEF DESCRIPTION OF THE DRAWINGS

FIG. 1 is an elevational diagram showing the anatomical relationship of the jaws of a patient upon which teeth may be moved by the method and apparatus of embodiments of the present invention.

FIG. 2A illustrates in more detail the patient's lower jaw of FIG. 1 and provides a general indication of how teeth may be moved by the method and apparatus of embodiments of the present invention.

FIG. 2B illustrates the jaw of FIG. 1 together with an incremental position adjustment aligner which has been configured according to the methods and apparatus of embodiments of the present invention.

FIG. 3 is a chart illustrating the peel strength of an exemplary PSA composition having a 1:2.5 ratio of polymer to tackifier resin formulation, in accordance with one embodiment of the present invention.

FIG. 4A is a top-down view of a compliance indicator configured according to the methods and apparatus of embodiments of the present invention.

FIG. 4B is a related top-down and cross sectional view of a pressure sensitive adhesive application configured according to the methods and apparatus of embodiments of the present invention.

FIG. 4C is a cross-sectional view of the pressure sensitive adhesive applications of FIGS. 4A and 4B in accordance with embodiments of the present invention.

FIG. 4D is a side view of a pressure sensitive adhesive application showing a relationship between an object and a pressure sensitive adhesive layer in accordance with one embodiment of the present invention.

FIG. 4E is a cross sectional view of a compliance indicator with a backing layer in accordance with one embodiment of the present invention.

FIG. 5 is a flow chart illustrating an exemplary method for forming a button for a dental attachment in accordance with one embodiment of the present invention.

FIG. 6 is an illustration of a plurality of buttons located on a surface of a substrate, wherein the buttons are peelable for fixedly attaching to a polymeric dental appliance in accordance with one embodiment of the present invention.

FIG. 7 is a flow chart illustrating an exemplary method for attaching a compliance indicator in accordance with one embodiment of the present invention.

### DETAILED DESCRIPTION OF THE INVENTION

Reference will now be made in detail to the preferred embodiments of the present invention, moisture sensitive adhesives for orthodontic applications, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims.

Furthermore, in the following detailed description of the present invention, numerous specific details are set forth in order to provide a thorough understanding of the present invention. However, it will be recognized by one of ordinary skill in the art that the present invention may be practiced without these specific details. In other instances, well known methods, procedures, and components have not been described in detail as not to unnecessarily obscure aspects of the present invention.

Accordingly, various embodiments of the present invention disclose moisture resistant pressure sensitive adhesives for use within an oral fluids environment. As a result, embodiments of the present invention disclose pressure sensitive adhesives that are capable of strong adhesion, strong cohesion, and excellent oral fluids resistance properties for use in an oral environment. These adhesives are used to mount water soluble polymer compliance indicators onto polymeric dental appliances that are exposed to the oral fluids environment for extended periods without peeling or softening of the pressure sensitive adhesives.

### System and Method for Positioning Teeth

Orthodontic treatments involve repositioning misaligned teeth and improving bite configurations for improved cosmetic appearance and dental function. Repositioning is accomplished by applying gentle controlled forces to a patient's teeth over an extended period of time. Due to the limited space within the oral cavity and extensive movements that some teeth must undergo, the teeth will often be moved throughout a series of intermediate patterns to properly arrange the teeth. For example, molars may be temporarily moved backwards (distalized) to create adequate space for movement of the incisors. Thus, a single patient may experience an average of 25-30 stages or alignment patterns before achieving the final desired teeth arrangement.

Conventionally, repositioning of teeth has been accomplished by what are commonly referred to as “braces.” Braces comprise a variety of appliances such as brackets, bands, archwires, ligatures, and O-rings. After they are bonded to the teeth, periodic meetings with the orthodontist are required to adjust the braces. This involves installing different archwires having different force-inducing properties or by replacing or tightening existing ligatures. Between meetings, the patient may be required to wear supplementary appliances, such as elastic bands or headgear, to supply additional or extraoral forces. Although conventional braces are effective, their use is often a tedious and time consuming process and requires many visits to the orthodontist's office. Moreover, from the patient's perspective, the use of braces is unsightly, uncomfortable, presents a risk of infection, and makes brushing, flossing, arid other dental hygiene procedures difficult.

In embodiments of the present invention, repositioning of teeth may be accomplished with the use of a series of removable elastic positioning appliances, referred to as “aligners”. Such appliances comprise a thin shell of elastic polymeric material that generally conforms to a patient's teeth but is slightly out of alignment with an initial or immediately prior tooth configuration. Placement of the elastic aligner over the teeth applies controlled forces in specific locations to gradually move the teeth into the new configuration. Repetition of this process with successive aligners comprising new configurations eventually move the teeth through a series of intermediate arrangements to a final desired arrangement. Conveniently and advantageously, the appliances are not affixed and the patient may place and replace the appliances at any time during the alignment process.

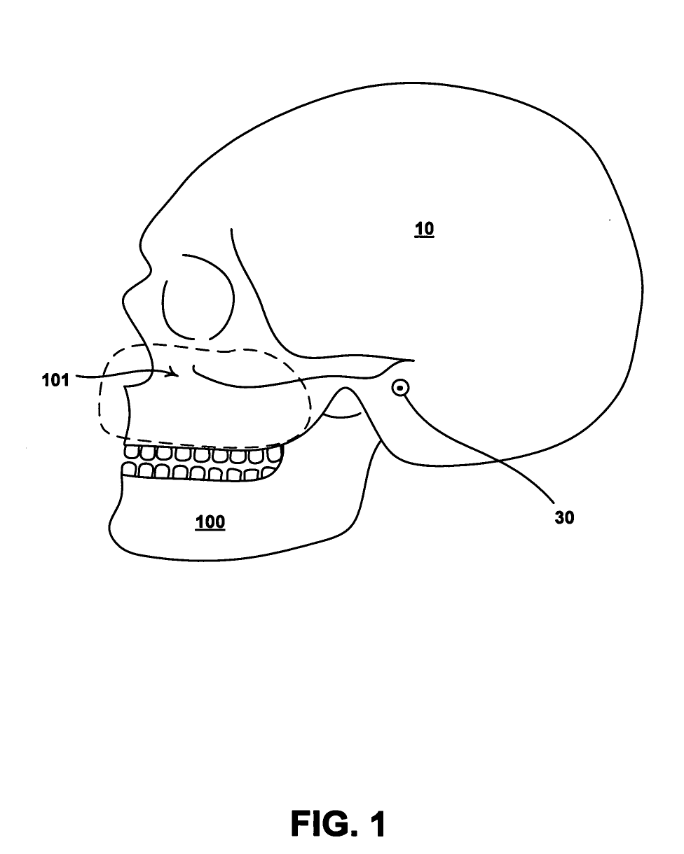

FIG. 1 is an illustration of a skull 10, upon which the repositioning of teeth is accomplished with a series of removable aligners, in accordance with one embodiment of the present invention. The skull 10 includes a maxilla or upperjaw 101 outlined approximately by the dotted circle. A set of upper teeth is associated with the upperjaw 101. The skull 10 also includes a mandible or lowerjaw 100. A set of lower teeth is associated with lowerjaw 100. Lower jaw 100 hinges at a joint 30 to skull 10. Joint 30 is called a temporomandibular joint (TMJ).

In one embodiment, a computer model ofjaws 100 and 101 is generated. A computer simulation is capable of modeling interactions among the teeth on jaws 100 and 101. The computer simulation allows the system to focus on motions involving contacts between teeth mounted on the jaws. The computer simulation allows the system to render realistic jaw movements which are physically correct when jaws 100 and 101 contact each other. The modeling of jaws 100 and 101 places the individual teeth in a treated position.

Further, the model can be used to simulate jaw movements including protrusive motions, lateral motions, and “tooth guided” motions where the path of lower jaw 100 is guided by teeth contacts rather than by anatomical limits ofjaws 100 and 101. Motions are applied to one jaw, but may also be applied to both jaws. Based on the occlusion determination, the final arrangement of the teeth in jaws 100 and 101 can be ascertained.

As a result, an initial digital data set (IDDS) representing an initial tooth arrangement and a final digital data set (FDDS) representing a final tooth arrangement are generated. Based on both the IDDS and the FDDS, a plurality of intermediate digital data sets (INTDDSs) are defined to correspond to incrementally adjusted aligners. The INTDDSs are defined using techniques for aligning teeth (e.g., the standard arch method, etc.). Thereafter, a set of incremental position adjustment aligners are produced based on the INTDDs and the FDDS. The aligners are designed to be worn over the teeth and to reposition the teeth to each of the tooth arrangements.

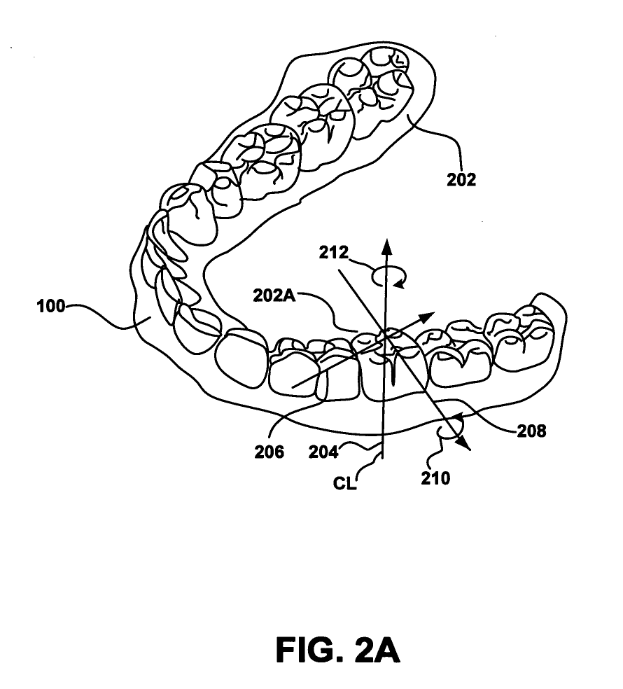

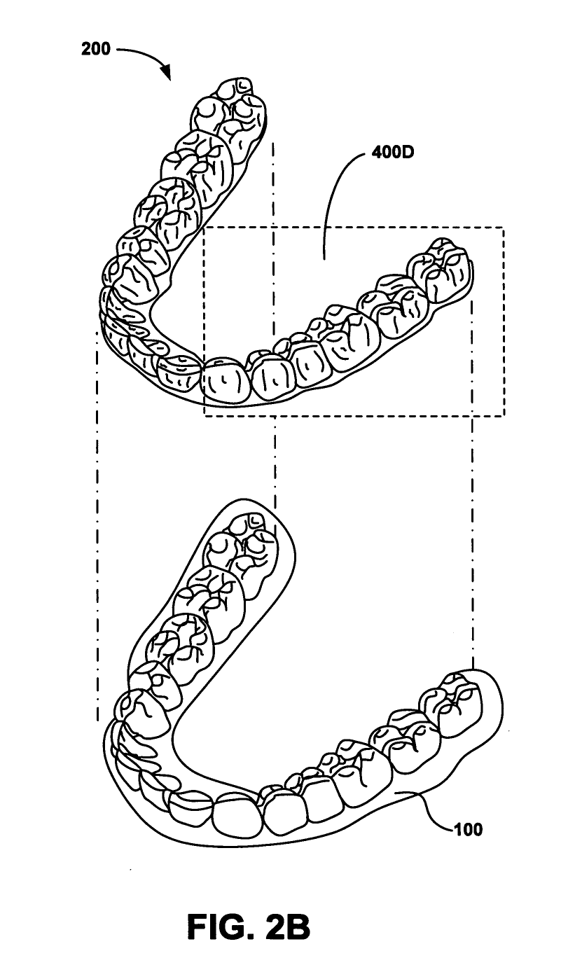

Referring now to FIG. 2A, lower jaw 100 of FIG. 1 includes a plurality of teeth, typically shown as 202, for example, and is provided as an illustration of the repositioning of teeth through the use of an aligner, in accordance with one embodiment of the present invention. The lower jaw 100 of FIG. 2A includes sixteen teeth in the plurality of teeth 202, to include exemplary tooth 202A. Embodiments of the present invention are capable of moving at least some of these plurality of teeth 202 from an initial tooth arrangement to a final tooth arrangement through a series of alignment stages.

In FIG. 2A, an arbitrary frame of reference is shown, and is used to describe how teeth in lower jaw 100 may be moved. For example, the frame of reference can be used to describe how tooth 202A is moved. An arbitrary centerline (CL) is drawn through exemplary tooth 202A. With reference to this centerline, the associated tooth may be moved in the orthogonal directions represented by axes 204, 206, and 208. As shown in FIG. 2A, axis 206 illustrates movement of tooth 202A between the anterior and posterior portions of lower jaw 100. Axis 210 illustrates side to side movement of tooth 202A in lower jaw 100. Axis 212 illustrates the upward and downward movements of tooth 202A in relation to lower jaw 100. In the configuration of FIG. 2A, the centerline corresponds to axis 204.

Additionally, the centerline may be rotated about the axis 208 (root angulation) and 204 (torque) as indicated by arrows 210 and 212, respectively. Also, the exemplary tooth 202A may be rotated about the axis 206. Thus, all possible free-form motions of tooth 202A can be performed.

A patient generally uses a repositioning aligner, corresponding to a given alignment stage in a treatment plan, until the aligner is no longer applying sufficient repositioning forces to the patient's teeth. When a patient first places an aligner over their teeth, the misalignment of the aligner with the teeth will apply forces on the teeth at the points of contact. The larger the misalignment, the stronger the repositioning force.

As the teeth gradually move into a desired arrangement for a given alignment stage and with the continued use of a corresponding aligner, the misalignment between the teeth and the aligner decreases and the applied force lessens until an equilibrium is reached. When the teeth substantially reach the desired configuration associated with the corresponding aligner, the force may approach zero. It is at this point that the useful life of such an aligner for applying repositioning force has ended. The patient may then progress to the next alignment stage in the treatment plan and begin wearing the next successive elastic repositioning aligner. The new aligner will apply repositioning forces to move the teeth to the next desired arrangement corresponding to the next alignment stage, repeating the aligner wear cycle.

In summary, the first aligner of a series of aligners will have a geometry selected to reposition a patient's teeth from the initial teeth arrangement to a first intermediate arrangement. After the first intermediate arrangement is approached or achieved, one or more additional, intermediate aligners will be successively placed on the teeth, where such additional aligners have geometries selected to progressively reposition teeth from the first intermediate arrangement through successive intermediate arrangement(s). The treatment will be finished by placing a final aligner in the patient's mouth, where the final aligner has a geometry selected to progressively reposition teeth from the last intermediate arrangement to the final tooth arrangement.

Referring now to FIG. 2B, a single adjustment aligner 200 which is worn by the patient in order to achieve an incremental repositioning of individual teeth in lower jaw 100 of FIG. 1 is shown, in accordance with one embodiment of the present invention. The aligner 200 is one of a series of incremental position adjustment aligners worn by the patient to realign teeth from an initial arrangement to a final arrangement. For brevity and clarity, exemplary aligner 200 is shown for incremental repositioning of teeth in lower jaw 100; however, other embodiments are well suited to the use of aligners for repositioning of teeth in either or both the upper and lower jaws of a patient.

The exemplary aligner 200 comprises a polymeric shell having a cavity shaped to receive and resiliently reposition teeth from one tooth arrangement to a successive tooth arrangement. The polymeric shell will preferably, but not necessarily, fit over all teeth present in the upper or lower jaw (e.g., lower jaw 100). Often, only certain one(s) of the teeth will be repositioned while others of the teeth will provide a base or anchor region for holding the repositioning appliance in place as it applies the resilient repositioning force against the tooth or teeth to be repositioned.

In complex cases, however, many or most of the teeth will be repositioned at some point during the treatment. In such cases, the teeth which are moved can also serve as a base or anchor region for holding the repositioning appliance. Additionally, the gum, palate, opposing arch, and skeletal implants taken alone or in combination can serve as anchor regions, thus allowing all or nearly all of the teeth to be repositioned simultaneously. Usually, no wires or other means will be provided for holding the aligner in place over the teeth. In some cases, however, it will be desirable or necessary to provide individual anchors on teeth with corresponding receptacles or apertures in the aligner 200 so that the aligner 200 can apply an upward force on the tooth which would not be possible in the absence of such an anchor.

The planning and fabrication of such aligners as an exemplary elastic polymeric positioning appliance is described in detail in U.S. Pat. No. 5,975,893, and in published PCT application WO 98/58596 which designates the United States and which is assigned to the assignee of the present application.

Systems of preformed aligners employing technology described in U.S. Pat. No. 5,975,893, are commercially available from Align Technology, Inc., Santa Clara, Calif., under the tradename, Invisalign System. Align Technology, Inc., is the assignee of the present application. The Invisalign System relies on designing and fabricating the aligners to be worn by the patient throughout treatment. The design of the aligners relies on computer modeling of a series of successive tooth arrangements, and the individual aligners are designed to be worn over the teeth and to reposition the teeth to each of the tooth arrangements. Usually, the set of aligners which is designed and fabricated at the outset of the treatment is able to successfully reposition the teeth to a final desired arrangement.

In embodiments of the present invention, the aligners may be planned and all individual aligners fabricated at the outset of treatment. As such, the aligners may thus be provided to the patient as a single package or system. The order in which the aligners are to be used will be clearly marked, (e.g. by sequential numbering) so that the patient can place the aligners over his or her teeth at a frequency prescribed by the orthodontist or other treating professional. Unlike braces, the patient need not visit the treating professional every time an adjustment in the treatment is made. While patients will usually want to visit their treating professionals periodically to assure that treatment is going according to the original plan, eliminating the need to visit the treating professional each time an adjustment is to be made allows the treatment to be carried out in many more, but smaller, successive steps while still reducing the time spent by the treating professional with the individual patient. Moreover, the ability to use polymeric shell appliances which are more comfortable, less visible, and removable by the patient, greatly improves patient compliance, comfort, and satisfaction.

### Pressure Sensitive Adhesives for Orthodontic Applications

Throughout the body of this Specification, the use of the terms “aligner” or “dental aligner” is synonymous with the use of the terms “appliance” and “dental appliance” in terms of dental applications. For purposes of clarity, embodiments of the present invention are hereinafter described within the context of the use and application of dental appliances, and more specifically “polymeric shell dental appliances,” or “dental appliances.”

A pressure sensitive adhesive (PSA) composition is disclosed having strong adhesion, strong cohesion, and excellent oral fluids resistance properties, in accordance with one embodiment of the present invention. The PSA composition of the present embodiment is capable of use within an oral environment for long periods of time (e.g., 20-22 hours per day for two consecutive weeks) without peeling or softening of the PSA composition.

In general, a PSA composition includes a balance between elastic and viscous properties. The viscous property of the PSA composition brings the PSA composition and an attaching substrate into contact so that adhesive bonds between the two are capable of being formed. Once the bonds are formed, the PSA composition must have elastic strength so as to allow the PSA composition to be mounted to the attaching substrate in a substantially permanent fashion. In a PSA composition, a base polymer provides the elastic properties, while a tackifier provides the viscous properties.

In the present embodiment, the PSA composition comprises a polymer base as one part of the PSA composition. The polymer base comprises an elastomer base, in one embodiment. In another embodiment, the polymer base comprises an acrylic base.

In particular, the elastomer base is considered a rubber base in one embodiment and provides unique properties suitable for forming a PSA composition for use in an oral environment. For instance, the elastomer base is resistant to water, or oral fluids. As such, the PSA composition comprising an elastomer base is also resistant to water or oral fluids, and is not susceptible to softening.

Specifically, in one embodiment, the elastomer base comprises a styrenic block copolymer (SBC). The SBC rubber based polymer comprises a tri-block SBC polymer (SIS). The SIS copolymer comprises styrene end-blocks and an isoprene mid-block.

The mid-block isoprene domain provides adhesion or adhesive strength through tackification with the tackifiers, as will be described below. Adhesion strength provides for the adhesion or bonding of the PSA composition with the attaching substrates. As such, the PSA composition of the present embodiment provides for strong adhesion of the PSA composition to a polymeric dental appliance within an oral environment.

The styrene end-blocks provide for proper cohesive strength. Cohesive strength provides for good bonding between the PSA components. That is, the styrene end-blocks provide for holding the PSA components together. In one embodiment, the cohesive strength provided by the styrene end-blocks is achieved through domain interaction.

In another embodiment, the polymer base comprises an acrylic base. The acrylic based polymer that forms the PSA composition may not be as water or moisture repellant as the SBC, rubber based polymer previously described. However, for purposes of the present invention, the acrylic based, PSA composition of the present embodiment has water or moisture repellant characteristics that are well suited for use in an oral environment.

In one embodiment, the tackifier in the PSA composition comprises at least one bio-compatible hydrophobic tackifier. In this manner, the bio-compatible nature of the tackifier allows for safe use of the PSA composition within an oral environment. Also, the hydrophobic nature of the tackifier allows for formulated PSA composition adhesives to have water or moisture repelling characteristics, in accordance with one embodiment of the present invention.

Embodiments of the present invention disclose the use of SBC polymers that are formulated with hydrophobic tackifiers. The hydrophobic nature of these SBC polymers eliminates peeling and softening problems associated with conventional rosin based tackifiers that introduce hydrophilic properties to the overall PSA composition. Hence the resulting PSA composition of embodiments of the present invention are suitable for prolonged use in an oral environment.

In one embodiment, the hydrophobic tackifier comprises a hydrocarbon tackifier. As such, the SBC polymers that are formulated with hydrocarbon tackifiers allow the formulated PSA composition to be substantially water and moisture repellant. As such, the SBC polymers using hydrocarbon tackifier for forming the PSA composition would be suitable for use in an oral environment, as the PSA composition would not be susceptible to peeling and softening. Specifically, the PSA composition is capable of adhering strongly to polymeric dental appliances.

While embodiments of the present invention disclose a PSA composition for use within an oral environment, other embodiments of the present invention are well suited for purposes of adhesion to surfaces other than within an oral environment. For instance, the PSA compositions of embodiments of the present invention are suited for adhesion to surfaces used within any moisture laden environment. In one embodiment, the moisture resistant PSA composition comprises a polymer base and at least one hydrophobic tackifier that comprises a hydrocarbon tackifier. The PSA composition is structured for use in a moisture laden environment. In one particular embodiment, a total weight of the combination of the polymer base and the hydrophobic tackifier comprises a concentration of the polymer base comprising approximately 29 to 50 percent of the total weight of the combination, and a concentration of the at least one hydrophobic tackifier comprising approximately 28 to 50 percent of the total weight of the combination.

Table 1 provides an exemplary list of various adhesive compositions, in accordance with embodiments of the present invention. As shown in Table 1, each of the exemplary PSA compositions comprises an SIS polymer elastomer base and at least one hydrocarbon tackifier as the hydrophobic tackifier. Specifically, as shown in Table 1, the Kraton® D113P (SIS) polymer is selected as the base polymer for each of the PSA compositions in some embodiments of the present invention. Also, each of the PSA compositions include one or more of the Wingtack® 95 and 10 Aliphatic C5 hydrocarbon tackifiers of the Sartomer Company, in some embodiments. The exemplary PSA compositions of Table 1 provide excellent moisture resistant characteristics since both the polymer base and the selected tackifiers exhibit hydrophobic characteristics.

The PSA compositions of Table 1 are intended to be exemplary compositions only, and are not exhaustive of the PSA compositions of embodiments of the present invention. Other PSA compositions are well suited to the intended purposes of orthodontic applications or use within an oral environment in accordance with embodiments of the present invention. For instance, PSA compositions of other embodiments comprise acrylic based PSA compositions, as previously described. Still other PSA compositions of other embodiments comprise other types of hydrophobic tackifiers, and still other hydrocarbon tackifiers.

The ratio of polymer and tackifier resins for the PSA compositions as shown in Table Ivaries from the following concentrations: (1:1); (1:1.5); (1:2); and (1:2.5). For instance the concentration of the polymer base comprises approximately 29 to 50 percent of the total weight of a combination of the polymer base and the bio-compatible hydrophobic tackifier. Also, the concentration of the one or more bio-compatible hydrophobic tackifiers comprises approximately 28 to 50 percent of the total weight of the combination of the polymer base and the bio-compatible hydrophobic tackifier. Four exemplary PSA compositions are shown below in Table 1.

TABLE 1
Adhesive Compositions

| | | | WEIGHT |
|---|---|---|---|
| PSA COMPOSITION | INGREDIENTS | DESCRIPTION | PERCENTAGE |
| PSA Composition 1 | Kraton ® D1113P | SIS Polymer | 50 |
| | Wingtack ® 86 | Aliphatic C5 | 50 |
| | | Hydrocarbon Tackifier | |
| PSA Composition 2 | Kraton ® D1113P | SIS Polymer | 40 |
| | Wingtack ® 86 | Aliphatic C5 | 60 |
| | | Hydrocarbon Tackifier | |
| PSA Composition 3 | Kraton ® D1113P | SIS Polymer | 33 |
| | Wingtack ® 95 | Aliphatic C5 | 33 |
| | | Hydrocarbon Tackifier | |
| | Wingtack ® 10 | Aliphatic C5 | 33 |
| | | Hydrocarbon Tackifier | |
| PSA Composition 4 | Kraton ® D1113P | SIS Polymer | 29 |
| | Wingtack ® 95 | Aliphatic C5 | 43 |
| | | Hydrocarbon Tackifier | |
| | Wingtack ® 10 | Aliphatic C5 | 28 |
| | | Hydrocarbon Tackifier | |

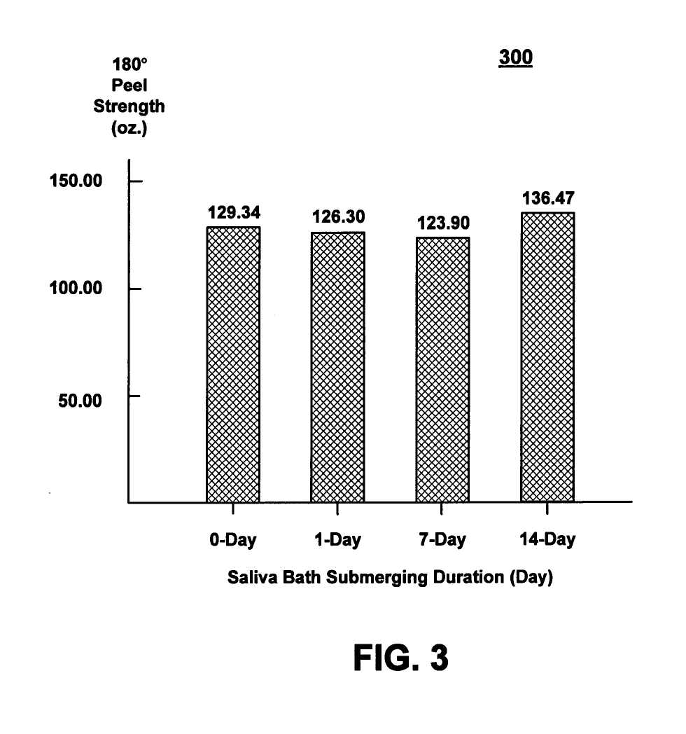

FIG. 3 is a chart 300 illustrating the adhesion performance of the PSA composition 4 of Table 1, in accordance with one embodiment of the present invention. The PSA composition 4 of Table 1 obtained approximately a 1:2.5 ratio of polymer to tackifier resin formulation. In one embodiment, the PSA composition 4 of Table 1 obtained the highest 180 degree peel strength of the four PSA compositions of Table 1.

As shown in FIG. 3, the PSA composition 4 has excellent moisture resistance stability. The chart 300 shows the peel strength of PSA composition 4 over a saliva submersion period of 14 days. Throughout the entire 14 day period, the peel strength is stable, ranging from approximately 129 on day-0 to 136 on day-14. As such, chart 300 indicates that PSA composition 4 is suitable for attaching buttons (e.g., compliance indicators) to polymeric dental appliances that are used within an oral environment for prolonged periods.

In accordance with another embodiment of the present invention, the PSA composition comprises a polymer base, a hydrophobic tackifier, and a solvent used in a process for creating the PSA composition. Table 2 provides an exemplary PSA composition comprising at least one solvent used for creating the PSA composition. Further, as shown in Table 2, the exemplary PSA composition also comprises an SIS polymer elastomer base and at least one hydrocarbon tackifier as the hydrophobic tackifier. Specifically, as shown in Table 2, the Kraton® D113P (SIS) polymer is selected as the base polymer. Also, the PSA composition of Table 2 comprises one or more of the Wingtack® 95 and 10 Aliphatic C5 hydrocarbon tackifiers from the Sartomer Company, in some embodiments. Also, the PSA composition of Table 2 has a 1:2.5 ratio of polymer to tackifier resin formulation. The exemplary PSA composition of Table 2 provides excellent moisture resistant characteristics since both the polymer base and the selected tackifiers exhibit hydrophobic characteristics.

TABLE 2
Adhesive Composition in Solvent Process

| | | | WEIGHT |
|---|---|---|---|
| PSA COMPOSITION | INGREDIENTS | DESCRIPTION | PERCENTAGE |
| PSA Composition | Kraton ® D1113P | SIS Co-Polymer | 12.87 |
| | Wingtack ® 95 | Aliphatic C5 | 19.3 |
| | | Hydrocarbon Tackifier | |
| | Wingtack ® 10 | Aliphatic C5 | 12.87 |
| | | Hydrocarbon Tackifier | |
| | n-heptane | Hydrocarbon Solvent | 46.22 |
| | Toluene | Solvent | 8.75 |

In accordance with another embodiment of the present invention, the PSA composition comprises a polymer base, a hydrophobic tackifier, and a phenolic antioxidant used in a hot-melt process for creating the PSA composition. Table 3 provides an exemplary PSA composition comprising at least one phenolic antioxidant used for creating the PSA composition. Further, as shown in Table 3, the exemplary PSA composition also comprises an SIS polymer elastomer base and at least one hydrocarbon tackifier as the hydrophobic tackifier. Specifically, as shown in Table 3, the Kraton® D113P (SIS) polymer is selected as the base polymer, in one embodiment. Also, the PSA composition of Table 2 comprises one or more of the Wingtack 95 and 10® Aliphatic C5 hydrocarbon tackifiers of the Sartomer Company. The PSA composition of Table 3 has a 1:2.5 ratio of polymer to tackifier resin formulation. The exemplary PSA composition of Table 3 provides excellent moisture resistant characteristics since both the polymer base and the selected tackifiers exhibit hydrophobic characteristics.

TABLE 3
Adhesive Composition in Hot-Melt Process

| | | | WEIGHT |
|---|---|---|---|
| PSA COMPOSITION | INGREDIENTS | DESCRIPTION | PERCENTAGE |
| PSA Composition | Kraton ® D1113P | SIS Co-Polymer | 28.29 |
| | Wingtack ® 95 | Aliphatic C5 | 42.43 |
| | | Hydrocarbon Tackifier | |
| | Wingtack ® 10 | Aliphatic C5 | 28.29 |
| | | Hydrocarbon Tackifier | |
| | Phenolic | Hydrocarbon Solvent | 1.00 |
| | Antioxidant | | |

Exemplary embodiments of the present invention are described within the context of attaching compliance indicators to dental attachments using the PSA compositions previously described that are suitable for use within an oral environment. While embodiments of the present invention are described within the context of attaching color indicators to dental attachments, other embodiments of the present invention are well suited to attaching any object using the PSA compositions described previously to dental attachments in a manner that is suitable for use within an oral environment.

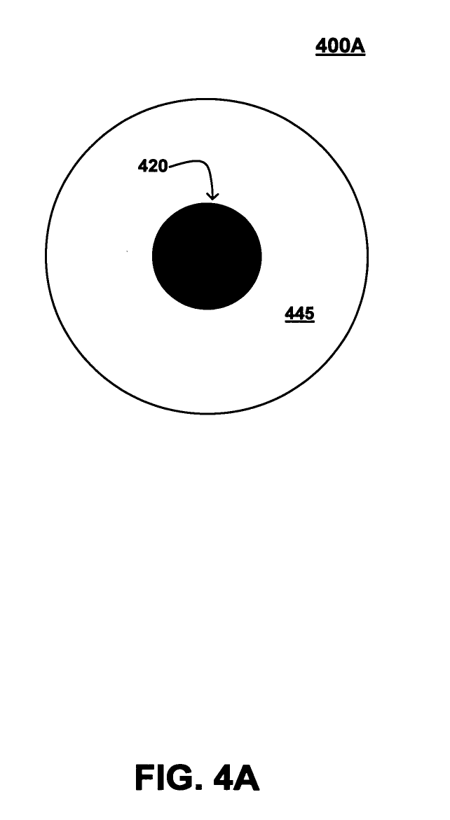

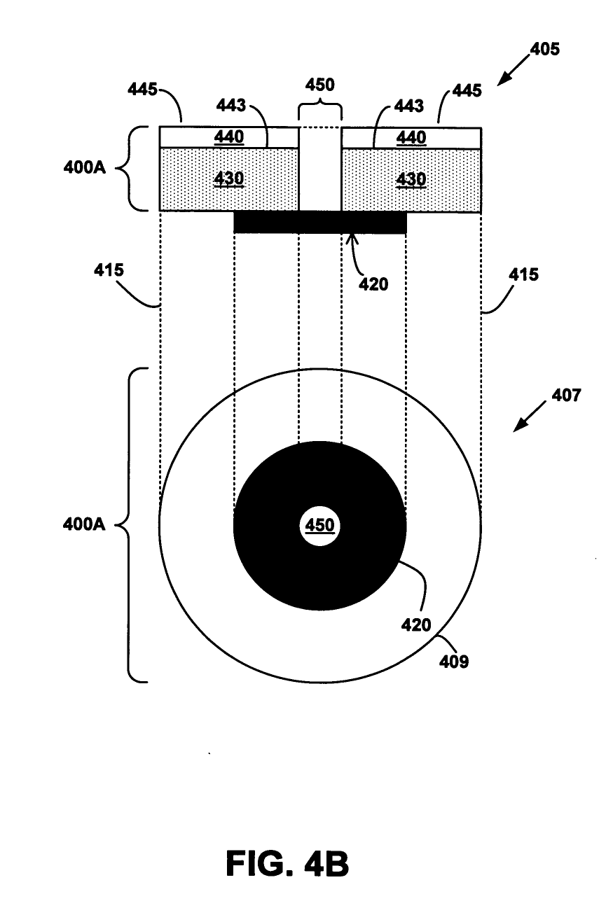

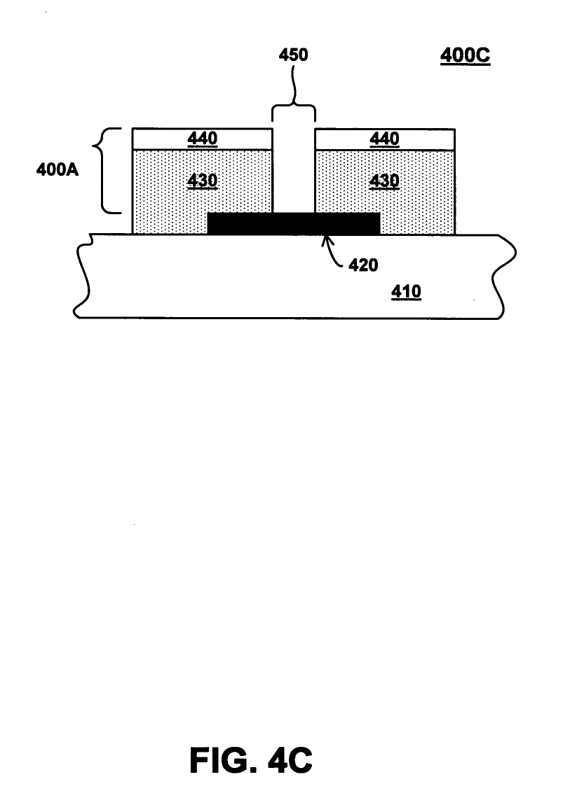

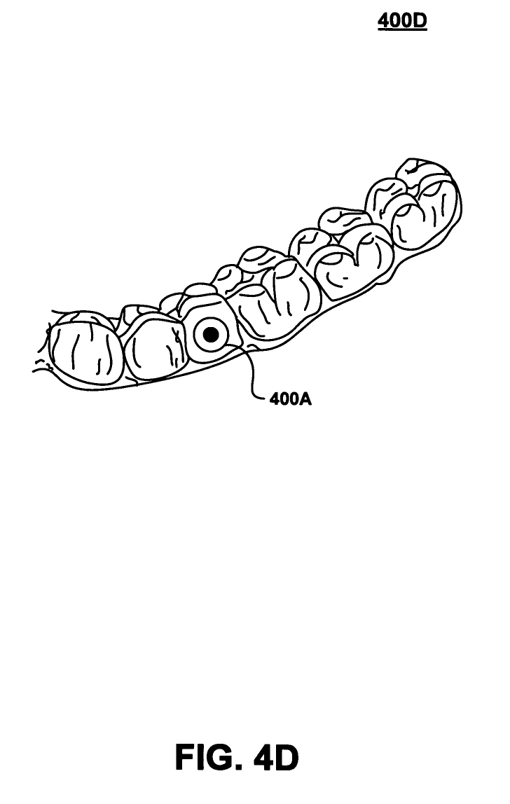

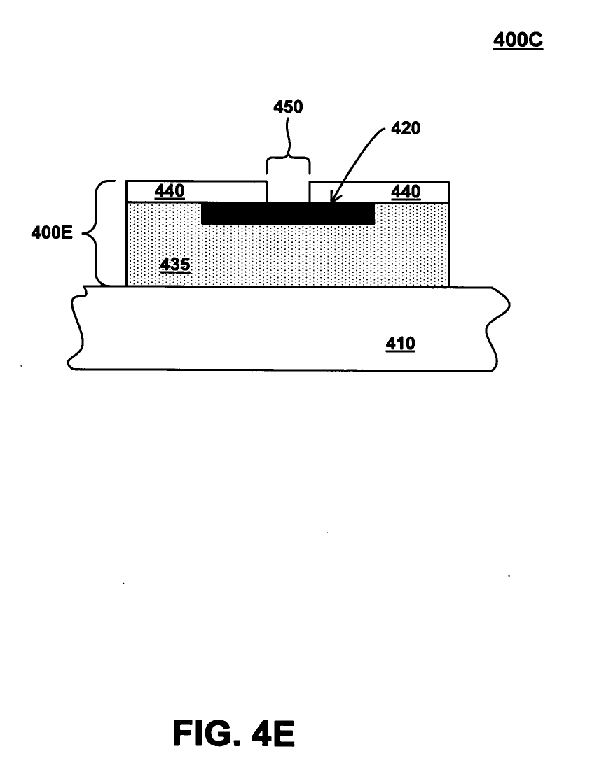

FIG. 4A is a top-down view of a compliance indicator 400A, in accordance with one embodiment of the present invention. Compliance indicator 400A comprises an indication attachment device, and when attached to a dental appliance is used to describe degrees of exposure to an oral environment. While the compliance indicator 400A as shown in FIG. 4A indicates a circular shape, other embodiments are well suited to compliance indicators that have different shapes, such as rectangular, triangular, oval, irregular, tooth shaped, etc.

Specifically, compliance indicator 400A comprises an object 420 that is visible through a top surface 445. In one embodiment, object 420 comprises a biodegradable polymer material that when exposed to an oral environment in a controlled manner is capable of indicating the amount of time the compliance indicator has been exposed to oral fluids within the oral environment. For instance, object 420 in compliance indicator 400A will change color, shape, size, etc. to indicate compliance. Further, while various other underlying layers may be visible in the top down view of compliance indictor 400A, for purposes of brevity and clarity, only object 420 is shown to be visible in FIG. 4A.

FIG. 4B provides relational cross sectional 405 and top-down 407 views of a pressure sensitive adhesive application illustrating the interaction between layers of compliance indicator 400A of FIG. 4A, including a pressure sensitive adhesive layer, in accordance with one embodiment of the present invention. Specifically, FIG. 4B shows compliance indicator 400A as a button for dental attachment that is suitable for use within an oral environment. Embodiments of the present invention are described within the context of using PSA compositions for attaching compliance indicators to dental appliances for use within an oral environment for purposes of illustration; however, it is understood that other embodiments are well suited to using PSA compositions for attaching objects to dental appliances for use within an oral environment.

Referring now to cross-sectional view 405 in FIG. 4B, compliance indicator 400A comprises a substantially water insoluble backing layer 440, in accordance with one embodiment of the present invention. Backing layer 440 comprises a top surface 445 and a bottom surface 443. Backing layer 440 provides for a platform from which to form compliance indicator 400A. That is, backing layer 440 supports the underlying layers that are coupled to backing layer 440. Various means are envisioned for coupling backing layer 440 to the underlying layers.

In one embodiment, backing layer 440 is substantially water insoluble. For instance, backing layer 440 would be substantially impermeable to oral fluids when placed within an oral environment. As such, backing layer 440 can be used to encapsulate and protect the underlying layers beneath backing layer 440 from exposure to the oral environment, when compliance indicator 400A is attached to a dental attachment.

In other embodiments, backing layer 440 is configured to provide for controlled exposure to the oral environment. For instance, as shown in FIG. 4A, an opening 450 is positioned within backing layer 440 in order to provide controlled exposure to oral fluids within an oral environment. Specifically, by varying the size, or diameter of opening 450, degrees of exposure to oral fluids can be achieved, in one embodiment. For instance, the larger the size of opening 450, more exposure to oral fluids can be achieved. The smaller the size of opening 450, less exposure to oral fluids can be achieved.

In still another embodiment, backing layer 440 is a semi-permeable membrane. In this case, the opening 450 is optional since backing layer 440 is able to control the exposure of oral fluids within the oral environment to the underlying layers beneath backing layer 440.

In one embodiment, backing layer 440 is substantially transparent. As such, objects and layers beneath backing layer 440 are visible through the backing layer 440. In one embodiment, an object 420 is visible through the backing layer 440. The object 420 may indicate varying degrees of exposure to the oral environment, as will be described more fully below. For example, as shown in FIG. 4B, top down view 407 illustrates an outline 409 of the compliance indicator 400A. Lines 415 illustrate outline 409 as the diameter of the compliance indicator. Since backing layer 440 is substantially transparent, backing layer 440 is not shown in top down view 407 within outline 409 of compliance indicator 400A, however object 420 is visible.

As shown in cross sectional view 405 of FIG. 4B, compliance indicator 400A comprises a pressure sensitive adhesive (PSA) layer 430 that is disposed on a back surface 443 of substantially water insoluble backing layer 440. Also, in top down view 407, lines 415 also illustrate the outer diameter of PSA layer 430 within outline 409. PSA layer 430 has strong adhesion, strong cohesion, and excellent oral fluids resistance characteristics, in accordance with embodiments of the present invention. Further, PSA layer 430 is bio-compatible with the human body, and is suitable for use within the oral environment. Because of the adhesive properties of PSA layer 430, compliance indicator 400A is capable of being fixedly attached to a dental appliance, in accordance with one embodiment of the present invention.

FIG. 4C is a cross-sectional diagram 400C illustrating the attachment of the compliance indicator 400A of FIGS. 4A and 4B to a dental appliance 410. As illustrated in FIG. 4C, in embodiments in accordance with the present invention, the chemical composition of PSA layer 430 comprises a polymer base that provides the elastic strength allowing the color indicator 400A comprising a PSA layer 430 to be mounted to dental appliance 410 in a substantially permanent fashion. For instance, in one embodiment, the polymer base comprises an elastomer base, and more specifically, a rubber base copolymer. In another embodiment, the polymer base comprises an acrylic base. Specifically, in one embodiment, the rubber base copolymer is resistant to water, or oral fluids typically found within an oral environment. Also, the polymer base is bio-compatible with the human body, in one embodiment.

The chemical composition of PSA layer 430 of FIGS. 4B and 4C also comprises at least one bio-compatible hydrocarbon tackifier. The bio-compatible nature of the tackifier allows for safe use of PSA layer 430 within an oral environment. The tackifier provides the viscous properties of PSA layer 430 so that the adhesive bonds between PSA layer 430 and dental appliance 410 are capable of being formed in order to attach PSA layer 430 to dental appliance 410.

Further, as previously described, the bio-compatible hydrocarbon tackifier is hydrophobic, and as such, PSA layer 430 has water or moisture repelling characteristics. In this case, compliance indicator 400A is suitable for use in an oral environment for extended periods of time, because PSA layer 430 is not susceptible to softening and peeling from dental appliance 410.

In one embodiment, PSA layer 430 is substantially transparent. As such, objects and layers beneath PSA layer 430 are visible. As shown in top down view 407 of compliance indicator 400A, layers beneath backing layer 440 and PSA layer 430 are visible. For instance, object 420 is visible through the substantially transparent backing layer 440 and PSA layer 430. Because the backing layer 440 and PSA layer 430 are substantially transparent, both layers 440 and 430 are not visible within outline 409 of compliance indicator 400A, however object 420 is visible.

The compliance indicator 400A also comprises an object 420 that is bonded to a surface of PSA layer 430. As shown in FIG. 4A, object 420 is bonded to the bottom surface of PSA layer 430, such that PSA layer 430 is sandwiched between backing layer 440 and object 420.

Returning back to FIG. 4C, in this configuration, when compliance indicator 400A is fixedly attached to dental appliance 410, object 420 is sandwiched between PSA layer 430 and dental appliance 410. As shown, PSA layer 430 surrounds object 420 such that exposure to object 420 is provided through opening 450, as previously described, in accordance with one embodiment of the present invention. More specifically, opening 450 is configured to be present in the top layers of compliance indicator 400A in order to expose object 420 to the oral environment. For instance, opening 450 is present in backing layer 440 and PSA layer 430. As such, object 420 is exposed to oral fluids through the opening 450 when dental appliance 410 is placed within an oral environment.

FIG. 4D is an illustration of the isolated region 400D of aligner 200 of FIG. 2B. In particular, FIG. 4D illustrates the compliance indicator 400A fixedly attached to the surface of aligner 200, also referred to as dental appliance for purposes of this invention. In embodiments of the present invention, compliance indicator 400A can be located at any location on the aligner 200 that is exposed to oral fluids in an oral environment.

In one embodiment, the compliance indicator 400A comprises an indication attachment device and indicates degrees of exposure to the oral environment. Specifically, object 420 implements the compliance features of compliance indicator 400A. In accordance with one embodiment of the present invention, object 420 comprises a biodegradable polymer material that is exposed to the oral environment in a controlled manner. In this case, the object 420 is capable of indicating the amount of time compliance indicator 400A has been exposed to oral fluids within an oral environment.

For example, after a predesigned period of use, the biodegradable polymer material in object 420 either changes shape, or size, or color, etc. As such, object 420 in compliance indicator 400A provides a compliance feature and indicates compliance by demonstrating a change in at least one characteristic of object 420, such as a change in color, shape, size, etc. In this manner, after the change in at least one characteristic, the compliance feature of compliance indicator 400A can visually indicate whether compliance indicator 400A has been exposed to the oral environment for the proper amount of time. Also, because the backing layer 440 and the PSA layer 430 are substantially transparent, the compliance feature of object 420 is readily visible. As such, when compliance indicator 400A is fixedly attached to a tooth or dental appliance 410, the compliance feature of compliance indicator 400A can visually indicate whether dental appliance 410 has been exposed to the oral environment for the proper amount of time.

In one particular embodiment, object 420 comprises a water soluble color indicator that is visible through the top surface 445 of the substantially water insoluble backing layer 440 and PSA layer 430. Opening 450 provides a controlled exposure to oral fluids when compliance indicator 400A is placed within the oral environment. For instance, after a predetermined period of time the water soluble color indicator in object 420 changes color (e.g., from blue to white). With controlled exposure of object 420 to oral fluids, because the water soluble color indicator in object 420 is visible through top surface 445, it is possible to determine whether compliance indicator 400A has been exposed to the oral environment for the predetermined period of time.

While the present embodiment discloses the use of opening 450 to expose object 420, as a compliance indicator, to oral fluids in the oral environment, other embodiments are well suited to various other techniques and systems for compliance indicators. A full discussion on the systems and techniques used for compliance indicators and their indications of usage for dental appliance compliance is described in detail in U.S. Publication No. 2006/0068353, which is assigned to the assignee of the present application.

In another embodiment of the present invention, the object 420 of FIGS. 4A, 4B and 4C comprises an image. In that case, the compliance indicator 400A comprises a button attachment used for purposes of decoration. For example, the image is a drawing of a character, or a slogan, or a picture, etc. The image in object 420 is visible through the substantially transparent layers that lie between object 420 and top surface 445. As such, in the present embodiment, the image as object 420 is visible through top layer 445 of backing layer 440. In this manner, the button comprising the image is attached directly to one or more teeth, or to the dental appliance 410. As such, the button 400A is worn as a decorative feature to show personal expression of the patient.

FIG. 4E is a cross sectional view of a pressure sensitive adhesive application showing an alternative relationship between object 420 and a pressure sensitive adhesive layer 435, in accordance with one embodiment of the present invention. Specifically, FIG. 4E illustrates the attachment of the compliance indicator 400E to dental appliance 410. The compliance indicator 400E is similar to compliance indicator 400A in FIGS. 4A, 4B, and 4C. For purposes of brevity and clarity, similarly numbered components in both FIGS. 4A, 4B, 4C, and 4D are intended to have similar features with similar functionalities.

As shown in FIG. 4E, compliance indicator 400E comprises a backing layer 440, in accordance with one embodiment of the present invention. Backing layer 440 is configured to provide for controlled exposure to an oral environment. In one embodiment, the backing layer 440 is water insoluble. Opening 450 is positioned in the backing layer 440 in order to expose object 420 to oral fluids in an oral environment. In another embodiment, backing layer 440 is a semi-permeable membrane. In this case, opening 450 is optional.

In one embodiment, backing layer 440 is substantially transparent. As such, objects and other layers beneath backing layer 440 are visible through backing layer 440. In the present embodiment, object 420 is visible through backing layer 440. In one embodiment, object 420 has compliance features as previously described and indicates varying degrees of exposure to the oral environment. As shown in FIG. 4E, object 420 is closer to top surface 445 of backing layer 440, thereby providing a less obstructive view of object 420 when compared to the embodiment of FIGS. 4B and 4C. That is, object 420 is visible immediately through top layer 445. In the present embodiment, other layers (e.g., PSA layer 435) below backing layer 440 need not be substantially transparent, since these layers do not block the view of object 420.

In addition, compliance indicator 400E comprises PSA layer 435 that is disposed on back surface 443 of backing layer 440. PSA layer 435 is analogous to PSA layer 430 of FIGS. 4B and 4C and has strong adhesion, strong cohesion, and excellent oral fluids resistance characteristics, and is bio-compatible with the human body. Because of the adhesive properties of PSA layer 435, compliance indicator 400E is capable of being fixedly attached to dental appliance 410, in one embodiment.

As shown in FIG. 4E, when compliance indicator 400E is fixedly attached to dental appliance 410, object 420 is sandwiched between backing layer 440 and PSA layer 435. As such, object 420 is encapsulated by backing layer 440 and PSA layer 435. Because of the hydrophobic qualities of backing layer 440 and PSA layer 435, exposure of object 420 to a fluid environment is controlled using opening 450, as previously described, in accordance with one embodiment of the present invention. More specifically, opening 450 is configured to be present in the top layers of compliance indicator 400D in order to expose object 420 to the oral environment. For instance, opening 450 is present in backing layer 440. As such, object 420 is exposed to oral fluids through the opening 450 when dental appliance 410 is placed within an oral environment.

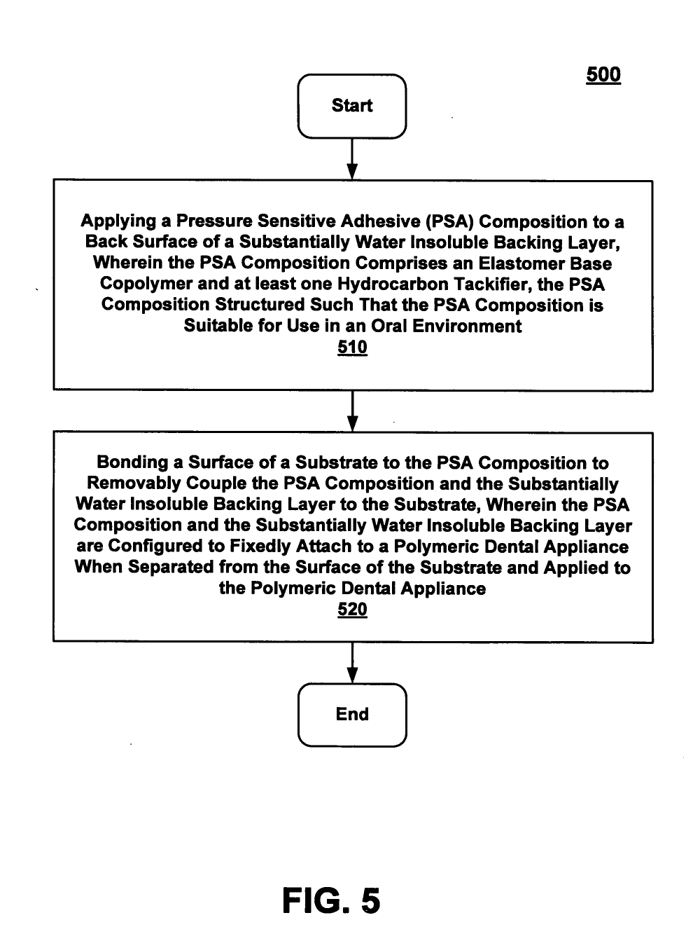

FIG. 5 is a flow chart 500 illustrating a method for forming a button for dental attachment, in accordance with one embodiment of the present invention. The method illustrated in flow chart 500 is suitable for creating compliance indicator 400A and 400E of FIGS. 4B, 4C, and 4E as previously described.

At 510, the present embodiment applies a PSA composition to a back surface of a backing layer. In one embodiment, the backing layer is substantially water insoluble. As previously described, the PSA composition has strong adhesion, strong cohesion, and excellent oral fluids resistance characteristics. In addition, the PSA composition of the present embodiment is suitable for use within an oral environment for long periods of time without peeling or softening of the PSA composition.

In the present embodiment, the PSA composition comprises an elastomer base and at least one hydrocarbon tackifier. Specifically, the elastomer base is resistant to water, or oral fluids. Specifically, in one embodiment, the elastomer base comprises a styrenic block copolymer (SBC). The SBC rubber based polymer comprises a tri-block SBC polymer (SIS). The SIS copolymer comprises styrene end-blocks and an isoprene mid-block. Further, the hydrocarbon tackifier comprises an aliphatic CS hydrocarbon tackifier, in one embodiment.

Specifically, in various implementations, the elastomer base copolymer comprises approximately 29 to 50 percent of a total weight of a combination of the elastomer base copolymer and the at least one hydrocarbon tackifier. Also, the at least one hydrocarbon tackifier comprises approximately 28 to 50 percent of the total weight of the combination of the elastomer base copolymer and the at least one hydrocarbon tackifier.

At 520, the present embodiment bonds a surface of a substrate to the PSA composition. In the manner, the PSA composition and the substantially water insoluble backing layer are removably coupled to the substrate. That is, the button that comprises the PSA composition and the backing layer are temporarily stored on a substrate. The button can be removed for further attachment to a polymeric dental appliance, in one embodiment. That is, the PSA composition and the substantially water insoluble backing layer are configured to fixedly attach to a polymeric dental appliance when separated from the surface of the substrate and applied to the polymeric dental appliance. In another embodiment, the PSA composition and the substantially water insoluble backing layer that comprise a button are configured to fixedly attach directly to one or more teeth when separated from the surface of the substrate and applied to the polymeric dental appliance.

In other embodiment, the method of flow chart 500 is implemented to create a decorative device that is attached to one or more teeth or a dental appliance. For instance, in this case, the object comprises an image, as previously described.

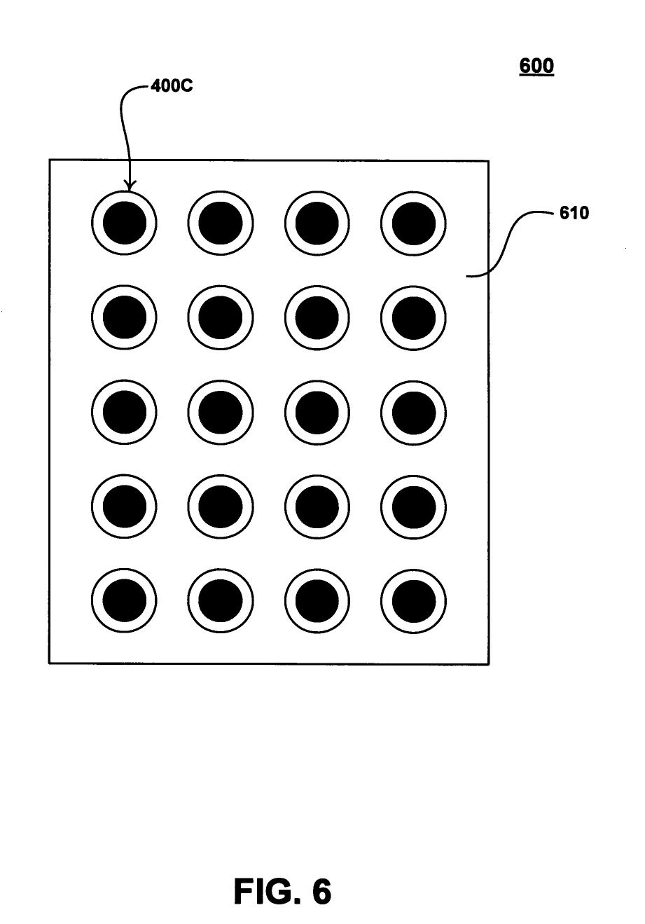

FIG. 6 is an illustration of a exemplary sheet 600 comprising a plurality of buttons, in accordance with one embodiment of the present invention. For instance, the button 400C of FIG. 4C in the plurality of buttons is shown attached to the substrate 610 for purposes of illustration. Each of the plurality of buttons is removably coupled to the substrate 610. That is, each of the buttons is capable of being removed from the substrate 610 and subsequently fixedly attached to a polymeric dental appliance, for example.

The sheet 600 is shown in a rectangular configuration, in the present embodiment, for purposes of illustration only. However, other embodiments are well suited to sheets 600 of various other configurations. For instance, the sheet may comprise a roll containing a plurality of buttons. As such, one button and the underlying substrate may be removed from the roll for subsequent ease of removal of the button from the substrate. Hence, embodiments of the present invention that are dispensed using substantially a sheet implementation allows a dental practitioner or patient to readily attach a button (e.g., compliance indicator) to a dental appliance, without requiring the time and labor intensive procedures of the conventional art in which adhesives used for attaching buttons are spread on the button and cured using ultraviolet light, for example. As such, button attachments of embodiments of the present invention are more convenient to use than buttons attached using conventional techniques.

In one embodiment, the method of flow chart 500 is implemented to create a compliance indicator. Various implementations of compliance indicators have been previously described. For instance, the button that is created comprises an object that comprises a water soluble color indicator. After a period of time, the object which is exposed to oral fluids in the oral environment changes (e.g., shape, color, or size, etc.) to indicate compliance with exposure of the button that includes the object to the oral environment.

One embodiment disposes the PSA composition applied to the back surface of the substantially water insoluble backing layer over an object. In addition, the backing layer and the PSA composition are substantially transparent. As such, the object is visible through a top surface of the substantially water insoluble backing layer. Specifically, in one embodiment, a compliance indicator as the object is disposed underneath the PSA composition and the substantially water insoluble backing layer. The compliance indicator is visible through a top surface of the substantially water insoluble backing layer. The compliance indicator indicates compliance by demonstrating a change in at least one characteristic of the compliance indicator, wherein the change in at least one characteristic comprises at least one of color, shape, and size. Further, the present embodiment, mounts the compliance indicator using the PSA composition to a dental appliance that is adapted to be worn over one or more teeth. As such, the compliance indicator is capable of indicating whether the dental appliance has been exposed to the oral environment for a predetermined period of time indicating compliance.

In another embodiment, the object is disposed between the PSA composition and the back surface of the substantially water insoluble backing layer. Since the backing layer is substantially transparent, the object is visible through a top surface of the substantially water insoluble backing layer. Specifically, in one embodiment, a compliance indicator as the object is inserted between the PSA composition and the back surface of the substantially water insoluble backing layer. The compliance indicator is visible through a top surface of the substantially water insoluble backing layer. The compliance indicator indicates compliance by demonstrating a change in at least one characteristic of said compliance indicator, wherein said change in at least one characteristic comprises at least one of color, shape, and size. Further, the present embodiment, mounts the compliance indicator using the PSA composition to a dental appliance that is adapted to be worn over one or more teeth. As such, the compliance indicator is capable of indicating whether the dental appliance has been exposed to the oral environment for a predetermined period of time indicating compliance.

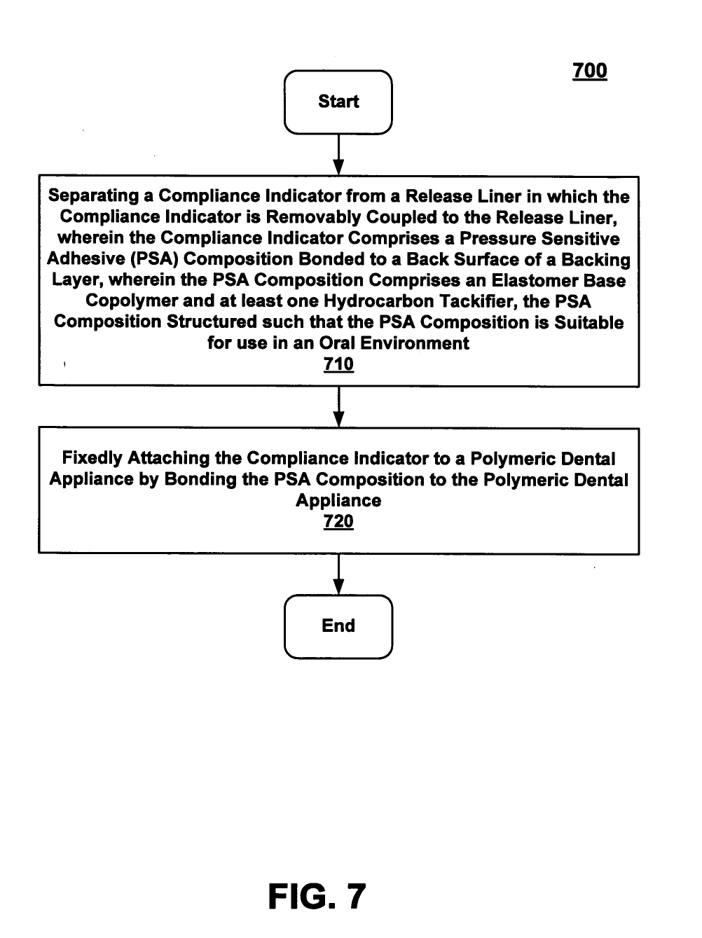

FIG. 7 is a flow diagram 700 illustrating steps in a method for attaching a compliance indicator in a dental application, in accordance with one embodiment of the present invention. The method illustrated in flow chart 700 is suitable for attaching the compliance indicators 400A and 400E of FIGS. 4A, 4B, 4C, and 4E as previously described.

At 710, the present embodiment separates a compliance indicator from a first substrate. In one embodiment, the first substrate comprises a release liner. The compliance indicator is removably coupled to the first substrate. That is, the compliance indicator is capable of being removed (e.g., peeled) from the first substrate (e.g., a peelable surface).

Also, the compliance indicator comprises a pressure sensitive adhesive (PSA) composition that is bonded to a back surface of a backing layer. The PSA composition comprises an elastomer base copolymer and at least one hydrocarbon tackifier. The PSA composition is structured to allow for attaching to the first substrate and subsequent attachment to a second substrate in a more permanent fashion. In particular, the PSA composition is structured such that the PSA composition is suitable for use in an oral environment.

At 720, after the removal of the compliance indictor from the substrate (e.g., release liner), the present embodiment provides for fixedly attaching the compliance indicator to a polymeric dental appliance. This is achieved by bonding the PSA composition to the polymeric dental appliance.

Compliance is determined when a characteristic of the compliance indicator changes. Specifically, the compliance indicator indicates compliance by demonstrating a change in at least one characteristic of said compliance indicator, wherein the characteristic comprises at least one of color, shape, and size, etc.

Accordingly, various embodiments of the present invention disclose moisture resistant pressure sensitive adhesives for use within an oral fluids environment. As a result, embodiments of the present invention disclose pressure sensitive adhesives that are capable of strong adhesion, strong cohesion, and excellent oral fluids resistance properties for use in an oral environment. These adhesives are used to mount water soluble polymer compliance indicators onto polymeric dental appliances that are exposed to the oral fluids environment for extended periods without peeling or softening of the pressure sensitive adhesives.

While the methods of embodiments illustrated in flow charts 500 and 700 show specific sequences and quantity of steps, the present invention is suitable to alternative embodiments. For example, not all the steps provided for in the method are required for the present invention. Furthermore, additional steps can be added to the steps presented in the present embodiment. Likewise, the sequences of steps can be modified depending upon the application.

Embodiments of the present invention, a system and method for moisture resistant pressure sensitive adhesives for use within an oral fluids environment have been described. While the invention is described in conjunction with the preferred embodiments, it is understood that they are not intended to limit the invention to these embodiments. On the contrary, the invention is intended to cover alternatives, modifications and equivalents, which may be included within the spirit and scope of the invention as defined by the appended claims. Furthermore, in the detailed description of the present invention, numerous specific details are set forth in order to provide a thorough understanding of the present invention. However, it will be recognized by one of ordinary skill in the art that the present invention may be practiced without these specific details. In other instances, well known methods, procedures, components, and circuits have not been described in detail as not to unnecessarily obscure aspects of the present invention.

1. A pressure sensitive adhesive (PSA) composition, comprising:
a polymer base; and
at least one bio-compatible hydrophobic tackifier, said PSA composition structured such that said PSA composition is suitable for use in an oral environment.

2. The PSA composition of claim 1, wherein said polymer base comprises an acrylic base.

3. The PSA composition of claim 1, wherein said polymer base comprises a elastomer base.

4. The PSA composition of claim 3, wherein said elastomer base comprises a rubber based copolymer.

5. The PSA composition of claim 4, wherein said rubber based copolymer comprises a styrenic block copolymer (SBC).

6. The PSA composition of claim 1, wherein said at least one bio-compatible hydrophobic tackifier comprises a hydrocarbon tackifier.

7. The PSA composition of claim 6, wherein said hydrocarbon tackifier comprises an aliphatic C5 hydrocarbon tackifier.

8. The PSA composition of claim 1, further comprising:
a solvent.

9. The PSA composition of claim 1, further comprising:
a phenolic antioxidant.

10. The PSA composition of claim 1, wherein a total weight of a combination of said polymer base and said at least one bio-compatible hydrophobic tackifier comprises:
a concentration of said polymer base comprising approximately 29 to 50 percent of said total weight of said combination; and
a concentration of said at least one bio-compatible hydrophobic tackifier comprising approximately 28 to 50 percent of said total weight of said combination.

11. A button for a dental attachment, comprising:
a substantially water insoluble backing layer;
a pressure sensitive adhesive (PSA) layer disposed on a back surface of said substantially water insoluble backing layer, wherein said PSA layer comprises:
a rubber base copolymer; and
at least one bio-compatible hydrocarbon tackifier, said PSA layer structured such that said PSA layer is suitable for use in an oral environment.

12. The button of claim 11, further comprising:
an object bonded to a surface of said PSA layer; and
wherein said surface of said PSA layer is configured for fixedly attaching to a polymeric dental appliance.

13. The button of claim 11, wherein said water insoluble backing layer and said PSA layer are substantially transparent.

14. The button of claim 13, wherein said object comprises a water soluble color indicator visible through a top surface of said substantially water insoluble backing layer.

15. The button of claim 13, wherein said object comprises a compliance indicator to indicate compliance by demonstrating a change in at least one characteristic of said compliance indicator, wherein said change in at least one characteristic comprises at least one of color, shape, and size.

16. The button of claim 13, wherein said object comprises an image visible through a top surface of said substantially water insoluble backing layer.

17. The button of claim 11, wherein said rubber base copolymer comprises a styrenic block copolymer (SBC).

18. The button of claim 11, wherein said at least one bio-compatible hydrocarbon tackifier comprises an aliphatic CS hydrocarbon tackifier.

19. The button of claim 11, wherein a total weight of a combination of said rubber base copolymer and said at least one bio-compatible hydrocarbon tackifier comprises:
a concentration of said rubber base copolymer comprising approximately 29 to 50 percent of said total weight of said combination; and
a concentration of said at least one bio-compatible hydrocarbon tackifier comprising approximately 28 to 50 percent of said total weight of said combination.

20. A method for forming a button for a dental attachment, comprising:
applying a pressure sensitive adhesive (PSA) composition to a back surface of a substantially water insoluble backing layer, wherein said PSA composition comprises an elastomer base copolymer and at least one hydrocarbon tackifier, said PSA composition structured such that said PSA composition is suitable for use in an oral environment; and
bonding a surface of a substrate to said PSA composition to removably couple said PSA composition and said substantially water insoluble backing layer to said substrate, said PSA composition and said substantially water insoluble backing layer configured to fixedly attach to a polymeric dental appliance when separated from said surface of said substrate and applied to said polymeric dental appliance.

21. The method of claim 20, further comprising:
disposing said PSA composition applied to said back surface of said substantially water insoluble backing layer over an object visible through a top surface of said substantially wafer insoluble backing layer.

22. The method of claim 20, further comprising:
inserting an object between said PSA composition and said back surface of said substantially water insoluble backing layer, said object visible through a top surface of said substantially water insoluble backing layer.

23. The method of claim 21, wherein said object comprises a water soluble color indicator.

24. The method of claim 21, wherein said object comprises an image.

25. The method of claim 20, wherein said elastomer base copolymer comprises a styrenic block copolymer (SBC).

26. The method of claim 20, wherein said at least one hydrocarbon tackifier comprises an aliphatic C5 hydrocarbon tackifier.

27. The method of claim 20, wherein said elastomer base copolymer comprises approximately 29 to 50 percent of a total weight of a combination of said elastomer base copolymer and said at least one hydrocarbon tackifier, and said at least one hydrocarbon tackifier comprises approximately 28 to 50 percent of said total weight of said combination.

28. The method of claim 20, further comprising:
inserting a compliance indicator between said PSA composition and said back surface of said substantially water insoluble backing layer, said compliance indicator visible through a top surface of said substantially water insoluble backing layer, wherein said compliance indicator indicates compliance by demonstrating a change in at least one characteristic of said compliance indicator, wherein said change in at least one characteristic comprises at least one of color, shape, and size; and
mounting said compliance indicator using said PSA composition to an appliance adapted to be worn over one or more teeth.

29. (canceled)

30. (canceled)

31. A moisture resistant pressure sensitive adhesive (PSA) composition, comprising:
a polymer base; and
at least one hydrophobic tackifier comprising a hydrocarbon tackifier, wherein said PSA composition is structured such that said PSA composition is suitable for use in a moisture laden environment, and wherein a total weight of a combination of said polymer base and said at least one hydrophobic tackifier comprises:
a concentration of said polymer base comprising approximately 29 to 50 percent of said total weight of said combination; and
a concentration of said at least one hydrophobic tackifier comprising approximately 28 to 50 percent of said total weight of said combination.
